(19) United States
(12) Patent Application Publication
(10) Pub. No.: US 2024/0041596 A1
(43) Pub. Date: Feb. 8, 2024

(54) VALVED CONDUIT IN A CONDUIT PROSTHESES

(71) Applicant: EDWARDS LIFESCIENCES CORPORATION, Irvine, CA (US)

(72) Inventors: Kyle W. Colavito, Flagstaff, AZ (US); Colin B. Heffner, Flagstaff, AZ (US)

(21) Appl. No.: 18/467,692

(22) Filed: Sep. 14, 2023

Related U.S. Application Data
(63) Continuation of application No. PCT/US22/22554, filed on Mar. 30, 2022.
(60) Provisional application No. 63/169,576, filed on Apr. 1, 2021.

(51) Int. Cl. A61F 2/24

(52) U.S. Cl. CPC A61F 2/2418; A61F 2/2415; A61F 2220/005; A61F 2210/0076; A61F 2220/0058

(57) ABSTRACT

Various aspects of the present disclosure are directed toward apparatuses, systems, and methods that include a valved conduit prosthesis including a valved conduit subassembly within a secondary conduit. The valved conduit subassembly include at least one leaflet that is coupled to an exterior surface of a primary conduit.

### CROSS REFERENCE TO RELATED APPLICATIONS

This application is a continuation of International Patent Application No. PCT/US2022/022554, filed Mar. 30, 2022, which claims the benefit of U.S. Patent Application No. 63/169,576, filed Apr. 1, 2021, the entire disclosures all of which are incorporated by reference for all purposes.

### FIELD

The present disclosure relates generally to prosthetic valves and more specifically to apparatuses, systems, and methods that include conduits having a valve structure therein.

### BACKGROUND

Bioprosthetic heart valves have been developed that attempt to mimic the function and performance of a native valve. Flexible leaflets may be coupled to a relatively rigid frame or other support structure that supports the leaflets and provides dimensional stability when implanted. The leaflets require some means for securing the leaflets to a support structure. In operation, the leaflets open when the upstream fluid pressure exceeds the downstream fluid pressure and close when the downstream fluid pressure exceeds the upstream fluid pressure. The leaflet free edges of the leaflets coapt under the influence of downstream fluid pressure closing the prosthetic heart valve to prevent downstream blood from flowing retrograde through the prosthetic heart valve.

Prosthetic heart valve durability under the repetitive loads of the leaflets opening and closing is dependent, in part, on the load distribution between the leaflet and the frame or support structure and, specifically, the attachment of the leaflet to the frame. Mechanical failure of the leaflet can arise, for example, at a mounting edge where the flexible leaflet is supported by the relatively rigid frame. The repetitive loads of leaflet opening and closing leads to material failure by fatigue, creep or other mechanism, depending in part on the leaflet material.

### SUMMARY

Described embodiments are directed to apparatus, system, and methods for valved conduit prostheses.

According to one example (“Example 1”), a valved conduit prosthesis includes a conduit having an interior surface and an exterior surface; and at least one leaflet having an external portion non-mechanically adhered to the exterior surface of the conduit and an internal portion arranged within the interior surface of the conduit.

According to another example (“Example 2”) further to Example 1, the external portion of the at least one leaflet is adhered to the exterior surface of the conduit by adhesive, thermal bonding, or chemical bonding.

According to another example (“Example 3”) further to any one of Examples 1-2, the conduit is free of sinuses.

According to another example (“Example 4”) further to any one of Examples 1-2, the conduit is free of mechanical coupling.

According to another example (“Example 5”) further to any one of Examples 1-4, the external portion of the at least one leaflet is attached to the exterior surface of the conduit, and the attachment is sutureless.

According to another example (“Example 6”) further to any one of Examples 1-5, the external portion of the at least one leaflet is adhered to the exterior surface of the conduit by a layer of adhesive film.

According to another example (“Example 7”) further to Example 6, wherein the adhesive film is arranged about a circumference of the conduit.

According to another example (“Example 8”) further to any one of Examples 6-7, further including a flexible film arranged about the circumference of the conduit and the adhesive film.

According to another example (“Example 9”) further to Example 8, the flexible film includes expanded Polytetrafluoroethylene (ePTFE) and the adhesive film comprises fluorinated ethylene propylene (FEP).

According to another example (“Example 10”) further to any one of Examples 8-9, further including a support frame coupled to the conduit by the flexible film.

According to another example (“Example 11”) further to Example 10, the support frame is formed of Polyether ether ketone (PEEK).

According to another example (“Example 12”) further to any one of Examples 1-11, further including at least one radiopaque markers arranged adjacent to the at least one leaflet on the exterior surface of the conduit.

According to another example (“Example 13”) further to any one of Examples 1-12, the interior surface of the conduit is diametrically constant and free of any macroscopic interruptions.

According to another example (“Example 14”) further to any one of Examples 1-13, the at least one leaflet is positioned within the conduit at a longitudinal location along the length of the conduit, and the conduit is diametrically constant at the longitudinal location where the at least one leaflet is positioned and through adjacent proximal and distal portions of the conduit.

According to one example (“Example 15”), a valved conduit prosthesis includes a conduit having an interior surface, an exterior surface, a proximal portion, and a distal portion; a leaflet attachment portion having an opening between the interior surface and the exterior surface of the conduit, and at least one leaflet having a conduit attachment section attached to the exterior surface of the conduit without mechanical alteration of the interior surface or the exterior surface of the conduit to mitigate against thrombus formation within the conduit.

According to another example (“Example 16”) further to Example 15, the at least one leaflet includes three leaflets, and the three leaflets are separated from one another within the interior of the conduit by lands.

According to another example (“Example 17”) further to Example 16, the conduit includes lands separating the leaflets at the conduit attachment section of each of the leaflets to form commissure gaps between the three leaflets within the interior surface of the conduit. In another example, the commissure gaps provide a regurgitant flow path when the valve is in the closed position.

According to another example (“Example 18”) further to any one of Examples 15-17, the conduit attachment section of the leaflet is attached to the exterior surface of the conduit by an adhesive, thermal bonding, or chemical bonding.

According to another example (“Example 19”) further to any one of Examples 15-18, the attachment section includes a first portion and a second portion, and the first portion is attached to the proximal portion of the exterior surface of the conduit, and the second portion is attached to the distal portion of the exterior surface of the conduit.

According to another example (“Example 20”) further to any one of Examples 15-19, the leaflet attachment portion is a portion of the conduit, and the leaflet attachment portion is denser than remaining portions of the conduit.

According to one example (“Example 21”), further to any one of Examples 15-20, the valved conduit prosthesis also includes a directional indicator on the exterior surface of the conduit to indicate the direction of blood flow within the conduit when in the open condition.

According to one example (“Example 22”), a method for reducing thrombus formation arising from the replacement of the native pulmonary valve or of a previously implanted pulmonary valved conduit prosthesis where partial or complete reconstruction of the right ventricular outflow tract and/or main pulmonary artery is desired, the method includes the steps of: providing a valved conduit prosthesis comprising a synthetic conduit having a distal end, proximal end, an interior, an exterior, and a leaflet attachment portion and at least one flexible synthetic leaflet having a portion external to the conduit and a portion internal to the conduit, wherein the leaflet portion external to the conduit, which is the conduit attachment portion of the leaflet, is attached to the exterior of the conduit at the attachment portion, and surgically implanting the valved conduit prosthesis.

According to one example (“Example 23”), a method for the replacement of the native pulmonary valve or of a previously implanted pulmonary valved conduit prosthesis where partial or complete reconstruction of the right ventricular outflow tract and/or main pulmonary artery is desired, the method includes the steps of: providing a valved conduit prosthesis comprising a synthetic conduit and at least one flexible synthetic valve leaflet attached to the synthetic conduit that has been rinsed in saline and has not been pre-clotted, and surgically implanting the valved conduit prosthesis.

According to one example (“Example 24”), a method for the replacement of the native pulmonary valve or of a previously implanted pulmonary valved conduit prosthesis where partial or complete reconstruction of the right ventricular outflow tract and/or main pulmonary artery is desired, the method includes the steps of: providing a valved conduit prosthesis that has been rinsed in saline and has not been pre-clotted, wherein said valved conduit prosthesis comprises a non-biological conduit and at least one flexible polymeric non-biological valve leaflet attached to the non-biological conduit, identifying the inflow and outflow portions of the valved conduit prosthesis, accessing the intended position with respect to the coronary arteries to assure there is no risk of coronary compression when implanted, optionally trimming the inflow and or outflow conduit, while under moderate tension, to the appropriate length for implantation, and attaching the valved conduit prosthesis.

According to one example (“Example 25”), a packaging insert for a valved conduit prosthesis, the packaging insert includes a support structure configured to fold to form one or more supports and to insert within the valved conduit prosthesis to support one or more leaflets within the valved conduit prosthesis.

According to an example (“Example 26”), a method of treating aortic valve disease by replacing the aortic root, comprising the steps of: providing a valved conduit prosthesis of any of Examples above and surgically implanting the valved conduit prosthesis.

According to an example (“Example 27”), the method of example 26, further comprising identifying an inflow portion and outflow portion of the conduit, accessing the intended position with respect to anatomy, optionally trimming the inflow portion and outflow portion of the conduit to the appropriate length for implantation, optionally outwardly tapering the inflow end or optionally everting and rolling the inflow portion toward the leaflet structure defining a sewing cuff, sectioning the ascending aorta, coupling the inflow portion of the valved conduit prosthesis to the left ventricle adjacent to or in the place of an excised aortic valve, and coupling the outflow portion of the valved conduit prosthesis to the sectioned ascending aorta.

According to an example (“Example 28”), the method of example 27, further comprising coupling coronary arteries to the outflow portion of the conduit 102 and establishing a flow path from the conduit lumen to the coronary arteries.

According to an example (“Example 29”), the method of example 28, further comprising coupling the coronary arteries to a sinus defined by the outflow portion.

According to an example (“Example 30), further to any of Examples 1-29, wherein the one or more leaflets comprise a composite material including a porous synthetic fluoropolymer membrane defining pores and an elastomer or elastomeric material filling the pores, and optionally TFE-PMVE copolymer comprising from 27 to 32 weight percent perfluoromethyl vinyl ether and respectively from 73 to 68 weight percent tetrafluoroethylene on at least a portion of the composite material, and optionally, the elastomer or elastomeric material comprises a TFE-PMVE copolymer, and optionally, the porous synthetic fluoropolymer membrane is ePTFE.

According to an example (“Example 31”), further to any of Examples 1-30, wherein the conduit has an inflow portion defining an inflow end and an outflow portion defining an outflow end, wherein the at least one leaflet is operable to open to allow flow from the inflow end to pass through the outflow end of the conduit in antegrade flow conditions, and is operable to close to restrict flow from flowing from the outflow end through the inflow end in retrograde flow conditions.

According to an example (“Example 32”), further to any of Examples 1-31, wherein the at least one leaflet comprises a composite material including a porous synthetic fluoropolymer membrane defining pores and an elastomer or elastomeric material filling the pores, and optionally TFE-PMVE copolymer comprising from 27 to 32 weight percent perfluoromethyl vinyl ether and respectively from 73 to 68 weight percent tetrafluoroethylene on at least a portion of the composite material, and optionally, the elastomer or elastomeric material comprises a TFE-PMVE copolymer, and optionally, the porous synthetic fluoropolymer membrane is ePTFE.

According to an example (“Example 33”), further to any of Examples 1-32, wherein the outflow portion defines a sinus adjacent to the at least one leaflet.

According to an example (“Example 34”), further to Example 33, wherein the sinus is operable for the surgical attachment of blood vessels and/or coronary arteries.

According to an example (“Example 35”), further to any one of Examples 1-34, wherein the inflow end defines an outward taper or is operable to be outwardly tapered.

According to an example (“Example 36”), further to any one of Examples 1-34, wherein the inflow portion is operable to be outwardly everted and rolled toward the valve structure defining a sewing cuff.

According to an example (“Example 37”), a valved conduit prosthesis includes a conduit having an interior surface defining a conduit lumen, an exterior surface, a proximal portion, and a distal portion, a leaflet attachment portion having an opening between the interior surface and the exterior surface of the conduit, and at least one leaflet having an attachment section attached to the exterior surface of the conduit, the at least one leaflet defining a valve structure.

According to an example (“Example 38”), further to Example 37, the at least one leaflet includes three leaflets, and the three leaflets are separated from one another within an interior of the conduit by commissure gaps.

According to an example (“Example 39”), further to Example 38, the conduit includes lands separating the leaflets at the attachment section of each of the leaflets to form the commissure gaps between the three leaflets within the interior surface of the conduit.

According to an example (“Example 40”), further to any one of Examples 37-39, the attachment section is attached to the exterior surface of the conduit by an adhesive, thermal bonding, or chemical bonding.

According to an example (“Example 41”), further to any one of Examples 37-40, the leaflet attachment portion is a portion of the conduit and the leaflet attachment portion is denser than remaining portions of the conduit.

According to an example (“Example 42”), further to any one of Examples 37-41, wherein the conduit has an inflow portion defining an inflow end and an outflow portion defining an outflow end, wherein the at least one leaflet is coupled to the conduit are operable to open to allow flow from the inflow end to pass through the outflow end of the conduit in antegrade flow conditions, and are operable to close to restrict flow from flowing from the outflow end through the conduit inflow end in retrograde flow conditions.

According to an example (“Example 43”), further to any one of Examples 37-42, the at least one leaflet comprises a composite material including a porous synthetic fluoropolymer membrane defining pores and an elastomer or elastomeric material filling the pores, and optionally TFE-PMVE copolymer comprising from 27 to 32 weight percent perfluoromethyl vinyl ether and respectively from 73 to 68 weight percent tetrafluoroethylene on at least a portion of the composite material, and optionally, the elastomer or elastomeric material comprises a TFE-PMVE copolymer, and optionally, the porous synthetic fluoropolymer membrane is ePTFE.

According to an example (“Example 44”), a valved conduit prosthesis comprises a secondary conduit having a secondary conduit inner surface defining a secondary conduit lumen therethrough, and a valved conduit subassembly including: a primary conduit having a primary conduit exterior surface and a primary conduit interior surface defining a primary conduit lumen and a slot therethrough, and at least one leaflet having an external portion coupled to the primary conduit exterior surface and an internal portion arranged within the primary conduit lumen so as to be operable as a one-way valve, the leaflet defining a valve structure, wherein the valved conduit subassembly is located within the secondary conduit lumen and coupled to the secondary conduit inner surface.

According to an example (“Example 45”), the valved conduit prosthesis of Example 44, wherein the external portion of the at least one leaflet is adhered to the primary conduit exterior surface by adhesive, thermal bonding, or chemical bonding.

According to an example (“Example 46”), further to Example 44, wherein the external portion of the at least one leaflet is coupled to the primary conduit exterior surface by a layer of adhesive film.

According to an example (“Example 47”), further to any one of Examples 44-46, wherein the valved conduit subassembly defines at least one step with the secondary conduit inner surface.

According to an example (“Example 48”), further to Example 47, wherein the at least one step is defined by the secondary conduit having a larger inner diameter compared with the inner diameter of the valved conduit subassembly.

According to an example (“Example 49”), further to any one of Examples 47-48, wherein the step may have a step height defined by the primary conduit inner surface and the secondary conduit inner surface at the step, wherein the step height is operable as a barrier to prevent pannus or other biological material from advancing into an upstream or downstream portion of the valved conduit subassembly.

According to an example (“Example 50”), further to any one of Examples 47-49, wherein the valved conduit subassembly defines an upstream length between the leaflet and the step, wherein the upstream length has a dimension of 0.1 mm to 3 mm, and a step height has a dimension of 0 mm to 3 mm.

According to an example (“Example 51”), further to any one of Examples 44-50, wherein the valved conduit has an inflow portion defining an inflow end and an outflow portion defining an outflow end, wherein the at least one leaflet is coupled to the primary conduit is operable to open to allow flow from the inflow end to pass through the outflow end in antegrade flow conditions, and are operable to close to restrict flow from flowing from the outflow end through the inflow end in retrograde flow conditions.

According to an example (“Example 52”), further to any one of Examples 44-51, wherein the at least one leaflet comprises a composite material including a porous synthetic fluoropolymer membrane defining pores and an elastomer or elastomeric material filling the pores, and optionally TFE-PMVE copolymer comprising from 27 to 32 weight percent perfluoromethyl vinyl ether and respectively from 73 to 68 weight percent tetrafluoroethylene on at least a portion of the composite material, and optionally, the elastomer or elastomeric material comprises a TFE-PMVE copolymer, and optionally, the porous synthetic fluoropolymer membrane is ePTFE.

According to an example (“Example 53”), further to any one of Examples 44-52, wherein the at least one leaflet includes more than one leaflets, and the more than one leaflets are separated from one another within the primary conduit inner surface of the primary conduit by commissure gaps.

According to an example (“Example 54”), further to Example 53, wherein the primary conduit includes lands separating the more than one leaflets at a conduit attachment section of each of the leaflets to form the commissure gaps between the three leaflets within the primary conduit inner surface of the primary conduit.

According to an example (“Example 55”), further to any one of Examples 44-54, wherein the valved conduit subassembly is attached to the secondary conduit inner surface by an adhesive, thermal bonding, or chemical bonding.

According to an example (“Example 56”), wherein a method of treating aortic valve disease by replacing an aortic root, comprising the steps of: providing a valved conduit prosthesis of any one of Examples 44-55; and surgically implanting the valved conduit prosthesis.

The foregoing Examples are just that, and should not be read to limit or otherwise narrow the scope of any of the inventive concepts otherwise provided by the instant disclosure.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1A is a side view of an example valved conduit prosthesis, in accordance with an embodiment;

FIG. 1B is an axial view of a valve structure in a closed configuration, in accordance with the embodiment of FIG. 1A;

FIG. 1C is an axial view of a valve structure in an open configuration, in accordance with the embodiment of FIG. 1A;

FIG. 2 is a side view of another valved conduit prosthesis, in accordance with another embodiment;

FIG. 3 is a cross-sectional view of another valved conduit prosthesis, in accordance with an embodiment;

FIG. 4A is a side view of a conduit including a cutting pattern for the conduit as used in a valved conduit prosthesis, in accordance with an embodiment;

FIG. 4B is a side view of a conduit including a cutting pattern for a conduit as used in a valved conduit prosthesis, in accordance with another embodiment;

FIG. 4C is a side view of a conduit including a cutting pattern for a conduit as used in a valved conduit prosthesis, in accordance with another embodiment;

FIG. 5 is a top view of a leaflet that may be used in a valved conduit prosthesis, in accordance with an embodiment;

FIG. 6A is an illustration of an example step for the attachment of a leaflet to a conduit, in accordance with an embodiment;

FIG. 6B is an illustration of another example step for the attachment of the leaflet to the conduit, in accordance with the embodiment of FIG. 6A;

FIG. 6C is an illustration of another example step for the attachment of the leaflet to the conduit, in accordance with the embodiment of FIGS. 6A-6B;

FIG. 7 is a cross-sectional view of a valved conduit prosthesis, in accordance with another embodiment;

FIG. 8 is a cross-sectional view of a valved conduit prosthesis, in accordance with another embodiment;

FIG. 9 is a side view of a valved conduit in a conduit prosthesis, in accordance with an embodiment;

FIG. 10 is a side cross-sectional view of the valve region of the embodiment of FIG. 9; and

FIG. 11 is a side cross-sectional view of the valve region of another embodiment.

### DETAILED DESCRIPTION

Persons skilled in the art will readily appreciate that various aspects of the present disclosure can be realized by any number of methods and apparatus configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

Although the embodiments herein may be described in connection with various principles and beliefs, the described embodiments should not be bound by theory. For example, embodiments are described herein in connection with prosthetic valved conduit prostheses. However, embodiments within the scope of this disclosure can be applied toward any valved conduit prosthesis, valve structure, or mechanism of similar structure and/or function. Furthermore, embodiments within the scope of this disclosure can be applied in non-cardiac applications.

Embodiments herein include various apparatuses, systems, and methods for a conduit having a valve structure operable as a prosthetic valve. The valve structure may include one or more leaflets operable as a one-way valve with the conduit defining a conduit lumen. The leaflet(s) open to permit flow and close to occlude the conduit lumen and prevent flow in response to differential fluid pressure.

The term “conduit”, as used herein, is defined as a tubular member having a lumen operable to direct fluid therethrough and having a wall that is impermeable to fluid transfer.

The terms “valved conduit” and “valved conduit prosthesis”, as used herein, is used interchangeably and are defined as a conduit with a valve structure that is coupled to and contained within the conduit for use in coronary or vascular procedures.

The term “valve structure”, as used herein, is defined as one or more separate leaflets or a leaflet construct having a plurality of leaflets that are coupled together that function as a one-way valve.

The term “leaflet construct”, as used herein, is defined as a valve structure comprising a plurality of leaflets that are coupled together with a commissure region between each leaflet.

The term “valved conduit assembly”, as used herein, is defined as a valved conduit of a reduced length that may be positioned within a primary conduit.

The term “membrane”, as used herein, refers to a sheet comprising a single material, such as, but not limited to, expanded fluoropolymer.

The term “composite material”, as used herein, refers to a combination of a membrane, such as, but not limited to, expanded fluoropolymer, and an elastomer or elastomeric material, such as, but not limited to, a fluoroelastomer. The elastomer or elastomeric material can be contained within a porous structure of the membrane, coated on one or both surfaces of the membrane, or a combination of coated on and contained within the porous structure of the membrane.

The term “laminate”, as used herein, refers to multiple layers of membrane, composite material, or other materials, such as elastomer or elastomeric material, and combinations thereof.

The term “film”, as used herein, generically refers to one or more of the membrane, composite material, or laminate.

term “biocompatible material”, as used herein, generically refers to any material with biocompatible characteristics including synthetic, such as, but not limited to, a biocompatible polymer, or a biological material, such as, but not limited to, bovine pericardium.

The term “coupled”, as used herein, means joined, connected, attached, adhered, affixed, or bonded, whether directly or indirectly, and whether permanently or temporarily

Embodiments herein include various apparatus, systems, and methods for a conduit having a valve structure operable as a prosthetic valve that can be used, such as, but not limited to, as a replacement of the native pulmonary valve or of a previously implanted pulmonary valved conduit prosthesis where partial or complete reconstruction of the right ventricular outflow tract and/or main pulmonary artery is desired. The valve structure may include one or more leaflets operable as a one-way valve with the conduit defining a conduit lumen. The leaflet(s) open to permit flow and close to occlude the conduit lumen and prevent reverse flow in response to differential fluid pressure. The conduit is operable to be surgically coupled to the corresponding anatomy with the use of, such as, but not limited to, suture.

Embodiments herein include various apparatus, systems, and methods for a conduit having a valve structure operable as a prosthetic valve that can be used, such as, but not limited to, replace an aortic valve and a portion of the aorta, such as the ascending aorta. The valve structure may include one or more leaflets operable as a one-way valve with the conduit defining a conduit lumen. The leaflet(s) open to permit flow and close to occlude the conduit lumen and prevent reverse flow in response to differential fluid pressure. The conduit is operable to be surgically coupled to the left atrium at a conduit proximal end and to a portion of the ascending aorta at a conduit distal end with the use of, such as, but not limited to, suture. In other embodiments, the conduit is also operable for the surgical attachment of one or more coronary arteries thereto to establish blood flow thereto.

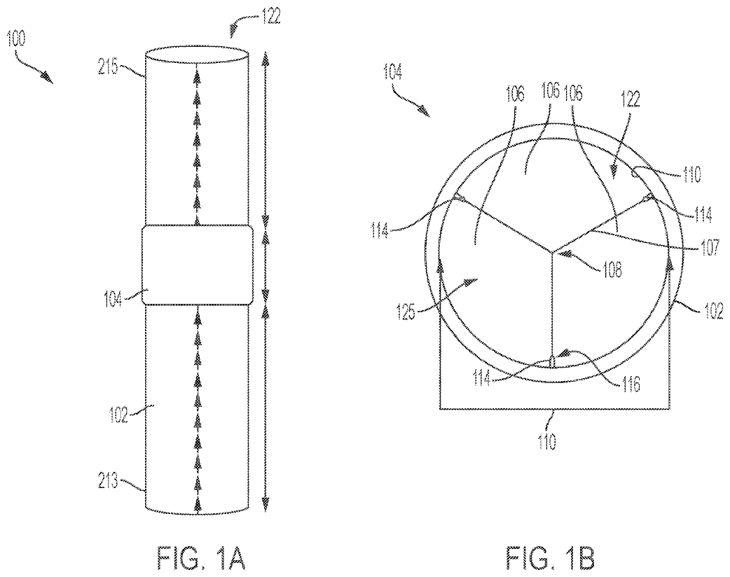

FIG. 1A is a side view of a valved conduit prosthesis boo, in accordance with an embodiment. The valved conduit prosthesis 100 includes a conduit 102 with a valve structure 104 arranged within the conduit 102. The conduit 102 includes an inflow end 213 and an outflow end 215 with a conduit lumen 122 extending therethrough such that the valve structure 104 is operable to allow flow in one direction from the inflow end 213 to the outflow end 215.

The valved conduit prosthesis 100 may be used, in a non-limiting example, to replace an aortic valve and at least a portion of the ascending aorta. In one non-limiting example, the valved conduit prosthesis 100 may be indicated for the correction or reconstruction of the aortic root and aortic valve, i.e., aortic root replacement, in pediatric patients. The valved conduit prosthesis 100 may also be indicated for the replacement of previously implanted homografts or valved conduits that have become dysfunctional or insufficient.

The valved conduit prosthesis 100 may be used, in a non-limiting example, as a shunt for connecting of the right ventricle to the pulmonary artery following a Norwood operation, as frequently performed for the treatment of hypoplastic left heart syndrome. In one non-limiting example, the valved conduit prosthesis 100 may be indicated for the correction or reconstruction of the right ventricle outflow tract (RVOT) in pediatric patients. Such reconstruction may be indicated for congenital heart disorders such as tetralogy of Fallot, Truncus Arterious, Dextro-Transposition of the Great Arteries, Pulmonary Atresia of Intact Ventricular Septum, or Aortic Valvular Disease. The valved conduit prosthesis boo may also be indicated for the replacement of previously implanted homografts or valved conduits that have become dysfunctional or insufficient. In addition, the valved conduit prosthesis 100 may have applications in treating a wider range of heart disorders, including other areas of the heart.

Generally, the term “distal” is used in the disclosure to refer to the outflow end 215 (distal end) or outflow direction of a valved conduit prosthesis 100, and in turn, the term “proximal” is used to refer to the inflow end 213 of a valved conduit prosthesis boo. Antegrade or forward flow is fluid flow from the inflow end 213 to the outflow end 215, and retrograde flow, also referred to as regurgitant flow when leaking through a closed valve structure 104, is fluid flow from the outflow end 215 to the inflow end 213.

In certain embodiments, the conduit 102 discussed herein includes an expanded fluoropolymer material made from porous ePTFE membrane. The expandable fluoropolymer, used to form the expanded fluoropolymer material described, can comprise PTFE homopolymer. In alternative embodiments, blends of PTFE, expandable modified PTFE and/or expanded copolymers of PTFE can be used. In certain embodiments the porous ePTFE membrane is rendered non-porous by, for example, a coating or imbibing, such as with an elastomer or elastomeric material, to prevent fluid from passing through the wall of the conduit 102, rendering the wall impermeable to fluid transfer.

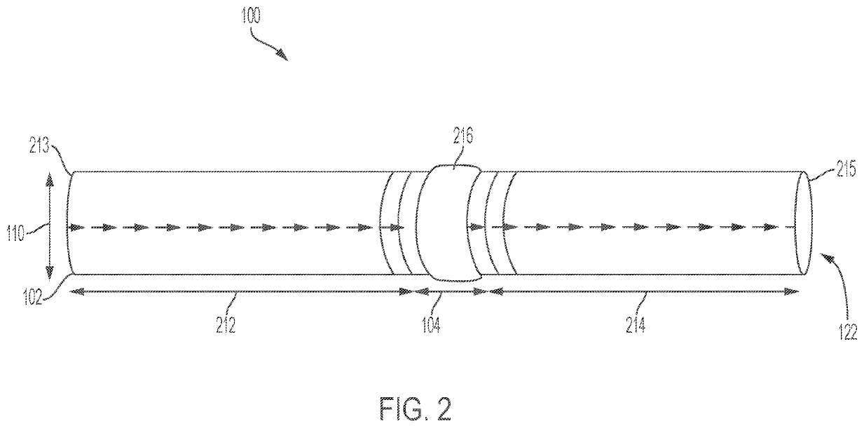

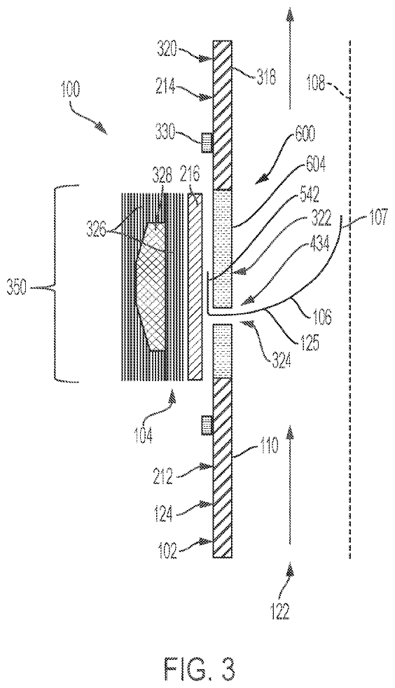

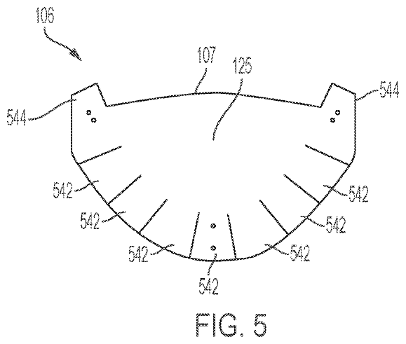

In certain embodiments, the entire conduit 102 may comprise the same material, such as ePTFE, but have different material properties at predetermined locations. As will be explained below and referenced in FIG. 3, by way of example, the conduit 102 at and adjacent to the leaflets 106 may be “densified”, that is, the material may be rendered more dense or less porous at that location by way of, but not limited to, a thermal, a coating or imbibing process. Such location of the conduit 102 at and adjacent to the leaflets 106 is referred herein as the valve region 350 as shown in FIG. The valve region 350 has a different material property than the remainder of the conduit 102 for a particular purpose, such as, but not limited to, providing a more rigid structure for supporting the leaflet attachment portion 322, to provide geometric integrity to the slits 434, for handling during manufacturing, and for strengthening the lateral cut 438 between the first conduit 102a and the second conduit 102b in embodiments having such elements, as shown in FIGS. 4C, 6A-6C.

FIGS. 1B and 1C illustrate an axial view of a valve structure 104 in a closed and open configuration, respectively, in accordance with the embodiment of FIG. 1A. The valve structure 104 includes leaflets 106 that extend into an interior of the conduit 102 from the interior surface 318 of the conduit 102 The valve structure 104 is defined as one or more separate leaflets 106 or a leaflet construct having a plurality of leaflets 106 that are coupled together that function as a one-way valve. Although three leaflets 106 are shown in FIGS. 1B and IC, the valve structure 104 may include one, two, three, four, or greater number of leaflets 106. As shown in FIG. 1B, the leaflets 106 close toward a center 108 of the conduit 102 in the closed configuration. In an open configuration, as shown in FIG. 1C, blood may flow through the valve structure 104 with the leaflets 106 being forced toward a conduit interior surface 110 of the conduit 102. In accordance with an embodiment, the leaflets 106 may be coupled as a valve structure 104 to the conduit 102 such that the conduit interior surface 110 of the conduit 102 has a smooth interior with a consistent interior diameter from the inflow end 213 to the outflow end 215 interrupted only by the leaflets 106 themselves. As will be described below, the leaflet 106 is coupled to the conduit 102 by way of tabs 542 that are coupled to the exterior surface 320 of the conduit 102 while the leaflet belly 125 extends into the conduit lumen 122 from the conduit interior surface 110.

In other embodiments, as provided below, a valved conduit assembly woo is provided, that is, a valved conduit prosthesis 100 is contained within a secondary conduit 1002. In some embodiments, a transition or step 218 is defined by a change of diameter between the valved conduit prosthesis 100 and the secondary conduit 1002 on the valved conduit interior surface no upstream and/or downstream of the leaflets 106 for a particular purpose. As further described below, the step 218 may prevent and pannus overgrowth or other blood or tissue product from encroaching onto the leaflet 106.

As further described with reference to FIG. 3, the conduit interior surface 110 of the conduit 102 remains smooth, that is, without a change in diameter up to and away from where the leaflets 106 protrude from the conduit interior surface no and into the conduit lumen 122. The conduit 102 defines slits 434 operable for the leaflet 106 to penetrate the conduit wall 124 and extend into the conduit lumen 122, while being coupled to the exterior surface 320 at a leaflet attachment portion 322 of the conduit 102.

One advantage of external attachment of the leaflet 106 is to minimize any flow disturbance and any resulting thrombus formation within the valved conduit prosthesis 100 that may occur with internal attachment accommodations that may disrupt the blood flow. By way of example, wherein if the attachment tabs 542 were coupled to the interior surface 318 of the conduit 102, the attachment tabs 542 would be exposed to the blood flow and cause a flow disturbance.

In certain embodiments, the conduit interior surface 110 of the conduit 102 is diametrically constant as shown in FIGS. 1B and 3. In addition, the leaflets 106 may be positioned within the conduit 102 at a longitudinal location along the length of the conduit 102 (e.g., as shown in FIG. 1A), and the conduit 102 is diametrically constant at the longitudinal location where the leaflets 106 are positioned and through adjacent proximal and distal portions of the conduit 102.

As shown in FIGS. 1B and 1C, a commissure gap 114 is located at each commissure 116 between a pair of leaflets 106. The commissure 116 is that location at the conduit interior surface no of closest adjacency of a pair of leaflets 106 at the leaflet free edges 107, as shown in FIGS. 4A-4C as lands 112 that define the commissure gap 114. The commissure gaps 114 allow retrograde flow through the conduit 102 when the leaflets 106 are closed. The retrograde flow may lessen the opportunity for blood to stagnate behind the leaflet 106, which can lead to thrombus formation. The commissure gaps 114 are sized such that leakage resulting from the retrograde flow is minimal and does not otherwise increase strain on the patient's heart to pump blood through the conduit 102. The commissure gaps 114 are associated with lands 112 in the conduit 102, as shown in detail in FIGS. 4A-4C, that is, that space between adjacent slits 434 at the closest adjacency.

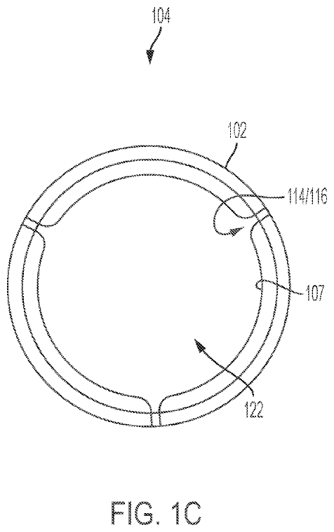

FIG. 2 is an illustration of another valved conduit prosthesis 100, in accordance with another embodiment. The valved conduit prosthesis 100 includes a conduit 102 and a valve structure 104. The conduit 102 includes an inflow portion 212 defining an inflow end 213 and an outflow portion 214 defining an outflow end 215. As indicate by the arrows on the conduit 102, the valve structure 104 is configured to allow blood flow through the conduit 102 from the inflow portion 212 to the outflow portion 214 and prevent blood flow from the outflow portion 214 to the inflow portion 212. The arrows may be a design feature printed on the conduit 102 to indicate the direction of blood flow within the conduit 102 to orient the physician for proper implantation. The arrows (a directional indicator) may be of a number of different shapes, sizes, lengths, or include other considerations.

As noted above with reference to FIG. 1B, the valve structure 104 includes one or more leaflets 106. The leaflets 106 are coupled to the conduit 102, and in combination with the conduit 102, defines a valve structure 104. The leaflet(s) 106 are coupled to an exterior surface 320 of the conduit 102 by a suitable means, such as, but not limited to, an adhesive, thermal bonding, and chemical bonding. In accordance with an embodiment, the leaflets 106 are attached, adhered, affixed, or bonded to the conduit 102 by an adhesive film 216, as shown in FIG. 3.

The adhesive film 216 may be a continuous or discontinuous layer wrapped about a circumference of the conduit 102. The adhesive film 216 may be placed, or wrapped about a circumference of the conduit 102, at a densified portion 604 (discussed with reference to FIG. 3) of the conduit 102 and/or beyond the densified portion 604 provided it is within the valve region 350 of the conduit 102 to couple the leaflets 106 to the exterior surface of the conduit 102. In other embodiments, the adhesive film 216 can be placed in the valve region 350 of the conduit 102 and also beyond the valve region 350 of the conduit 102 and may in some embodiments cover the entire conduit 102. The adhesive film 216 may seal the slits 434 from leaking blood therethrough. The adhesive film 216 may hold the two conduits together that are butt-joined at the leaflet, such as will be described as a first conduit 102a and a second conduit 102b as shown in FIGS. 4B and 4C.

Coupling or attaching the leaflet(s) to the exterior surface 320 of the conduit 102 by, for example, an adhesive, thermal bonding, or chemical bonding, as shown in FIG. 3, maintains a smooth interior surface 318 and consistent conduit inner diameter and maximized the effective valve orifice area (EOA) as compared with attachment arrangements that impact the profile of the interior surface 318.

FIG. 3 is a cross-sectional view of a valved conduit prosthesis 100, in accordance with an embodiment. The valved conduit prosthesis 100 includes a conduit 102 having an interior surface 318, an exterior surface 320, a proximal (or inflow) portion 212, and a distal (or outflow) portion 214. The conduit 102 includes a leaflet attachment portion 322 having an opening 324 between the interior surface 318 and the exterior surface 320 of the conduit 102. As described below, the opening 324 may correspond to the slits 434 as shown in FIGS. 4A-4C which may result is discontinuous slits 434 or a complete separation of a conduit 102 into a first conduit 102a and a second conduit 102b as shown in FIGS. 4B and 4C. The leaflet attachment portion 322 may be an integral portion of the conduit 102.

The valved conduit prosthesis 100 also includes a leaflet 106 that extends into the conduit 102 and toward the center 108 of the conduit. As shown in FIG. 3, the leaflet 106 is coupled to the exterior surface 320 of the conduit 102 via tabs 542. The leaflet 106 includes portions that are arranged external to the conduit 102, through the opening 324, and within the conduit 102 as shown in FIG. 3, with the leaflet belly 125 being defined as that portion of the leaflet 106 that extends within the conduit 102 from the interior surface 318. The leaflet 106 is coupled to the exterior surface 320 of the conduit 102 by an adhesive film 216, in accordance with this embodiment, shown by way of example. The adhesive film 216 may be overlaid or overlapped over the tabs 542 and/or over the slits 434 or openings 324. Other coupling means are anticipated, such as, but not limited to, thermal bonding, adhesive bonding, and mechanical coupling. The adhesive film 216 may be arranged within the bounds of the leaflet attachment portion 322. In addition, the leaflet attachment portion 322 may be densified, or have a material property that is more dense, as compared to the proximal (or inflow) portion 212 and the distal (or outflow) portion 214 of the conduit 102, identified as a densified portion 604.

The leaflet attachment portion 322 may be densified and/or rigidified relative to the rest of the conduit 102 for a particular purpose. By way of example, but not limited thereto, a densified leaflet attachment portion 322 may be provided such that the conduit 102 retains its shape at the valve region 350 during handling and use. By way of other examples, but not limited thereto, a densified leaflet attachment portion 322 may be provided to provide a smoother surface texture and/or a reduction in porosity so as to prevent flow disturbance and/or prevent tissue or pannus ingrowth. Densification refers to a process of selectively making the material more dense at selected locations, such as by heating and/or pressure and/or imbibing the pores with an elastomer or elastomeric material. In certain embodiments, the conduit 102 is formed from expanded polytetrafluoroethylene (ePTFE). For ePTFE material that may be relatively porous, the densification process will reduce porosity and/or make the area more rigid.

The valved conduit prosthesis 100 may also include a flexible film 326 arranged about the circumference of the conduit 102 and the adhesive film 216. The flexible film 326, in certain embodiments, may include one or more layers of the flexible film 326. The flexible film 326 may be wrapped multiple times about the conduit 102 and the adhesive film 216. The flexible film 326 may be wrapped as necessary to enhance the strength of the conduit 102 and/or the attachment of the leaflet 106 to the conduit 102 and/or for coupling the tabs 542 and/or for sealing the slits 434 or openings 324 from leaking, and/or to hold the two conduits together that are butt-joined at the leaflet 106, such as will be described as a first conduit 102a and a second conduit 102b as shown in FIGS. 4B and 4C. The adhesive film 216 may be placed or wrapped about a circumference of the conduit 102, at the leaflet attachment portion 322 (e.g., discussed with reference to FIG. 3) of the conduit 102 and/or beyond the leaflet attachment portion 322 provided it is within a valve region 35o of the conduit 102 to couple the leaflets 106 to the exterior surface 320 of the conduit 102. In other embodiments, the adhesive film 216 can be placed in the valve region 350 of the conduit 102 and also beyond the valve region 350 of the conduit 102 and may in some embodiments cover the entire conduit 102.

The flexible film 326, for example, enhances longitudinal tensile strength of the conduit 102 by adding column strength to the conduit 102. The flexible film 326, for example, may be used to ensure that the leaflet 106 is secured to the exterior surface 320 of the conduit 102. In certain embodiments and as noted above, the conduit 102 may be ePTFE. Particularly suitable are ePTFE vascular grafts having stretch/elastic behavior as they provide variable length with bend/kink resistance. In this regard, the exterior surface 320 of the conduit 102 can stretch to conform to the anatomy without kinking the interior surface 318 (luminal flow surface) and thus the conduit lumen 122 of the conduit 102. The flexible film 326 may also be ePTFE with the adhesive film 216 being fluorinated ethylene propylene (FEP). By using the flexible film 326 and the adhesive film 216 in combination, the leaflet 106 may be bonded (e.g., thermally) to the conduit 102.

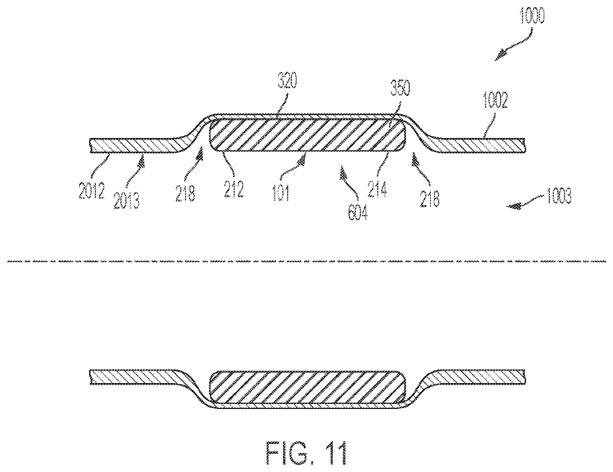

In certain embodiments, the valved conduit prosthesis 100 may also include a support frame 328 coupled to the exterior surface 320 of the conduit 102 by the flexible film 326, or by other means, such as thermal, adhesive, mechanical and frictional means. The support frame 328 can prevent compression, or otherwise reduce compressibility of the conduit 102 and the valve structure 104 resulting from anatomical compression forces and/or handling forces. In addition, the support frame 328, in certain embodiments, is formed of polyether ether ketone (PEEK). In these instances, the support frame 328 is not radiopaque, and therefore, allows a physician to better visualize the location of the leaflet 106 and the leaflet attachment portion 322 as compared if the support frame 328 was formed from other materials that would interfere with visualization. Visualizing the leaflet 106 and/or the leaflet attachment portion 322 may enhance the ability of a physician to accurately locate and place the conduit 102 in a target location. In other instances, the support frame 328 is formed of a radiopaque material as an aid to positioning or locating post-surgical implantation. In other instances, the support frame 328 is a metal, such as stainless steel, which may present a lower profile of the exterior surface 320 of the conduit 102 (e.g., being thinner), as shown in FIG. 11, while providing crush resistance, relative to another material.

The support frame 328 may or may not be radiopaque. In certain instances, the valved conduit prosthesis 100 may include one or more radiopaque markers 330 to assist in visualizing the valve region 350 of the conduit 102 post-procedure under fluoroscopic visualization. The one or more radiopaque markers 330 can be arranged adjacent to the leaflet 106 on the exterior surface 320 of the conduit 102. In certain embodiments and as shown in FIG. 3, the valved conduit prosthesis 100 may include radiopaque markers 330 on either side, longitudinally, of the leaflet 106 and the leaflet attachment portion 322 as well as may be placed in radial alignment with the leaflet 106, such as, but not limited to, the commissure 116, to aid in flow visualization and flow analysis. In this manner, the physician has markers for placement or localization of the leaflet 106 and the leaflet attachment portion 322 more particularly at the target location. The radiopaque markers 33o, in certain embodiments, are continuous or discontinuous ribbons of radiopaque material (e.g., gold) wrapped or placed about a circumference of the conduit 102.

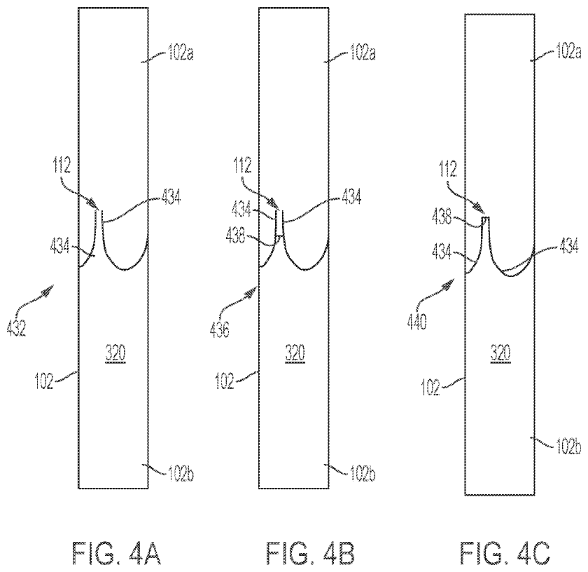

FIG. 4A is an illustration of a cutting pattern 432 for a conduit 102 as used in a valved conduit prosthesis 100, in accordance with an embodiment where a single conduit component is utilized. The cutting pattern 432 includes multiple separate slits 434 (e.g., creating the opening 324 shown in FIG. 3). The slits 434 correspond to the number of leaflets 106, shown in FIG. 3, that will be coupled to the conduit 102. As shown in FIG. 4A, the plurality of slits 434 defined by the cutting pattern 432 are not interconnected. Accordingly, the conduit 102 is not separated into multiple pieces during assembly and a single conduit component is utilized. Although two slits 434 are shown in FIG. 4A, the cutting pattern 432 may have additional slits (e.g., three slits as in a three leaflet tricuspid valve, four slits, etc.). The slits 434 may be formed by laser cutting, hand cutting, or other similar methods. In the embodiment shown in FIGS. 4A-4C, each slit 434 defines a parabolic shape wherein the corresponding leaflets 106 will have a parabolic shape where it penetrates the conduit 102 at the interior surface 318. In other embodiments, each slit 434 defines three sides of an isosceles trapezoid so as to define a leaflet 106 that has a flat base and two straight sides as visualized wherein the valve region 350 is longitudinally cut and laid open and flat. The tabs 542 and 544, shown in FIG. 5, may be passed through the slits 434 from inside of the conduit 102 and affixed to the exterior surface 320, such as shown in FIG. 6C.

The slits 434 are separated by a land 112 where two leaflets 106 come into close proximity defining a commissure 116. As noted above, the slits 434 correspond to the number of leaflets (not shown) that are coupled to the conduit 102. The land 112 corresponds to the commissure 116 (shown in FIG. 1B), that is, the separation between the leaflets 106 within the conduit 102. When the leaflets 106 are in the closed position (e.g., the valve structure is closed), there is a space, the commissure gap 114, between the leaflets 106 as shown in FIG. 1B. Likewise, then the leaflets 106 are in the open position, there is a space, the commissure gap 114, between the leaflets 106 as shown in FIG. 1C. The slits 434 and the lands 112 correspond to the number of leaflets that are coupled to the conduit 102. The commissure gap 114 allows blood to wash-out the region behind the leaflets 106 when the leaflet is in the closed position. The retrograde or regurgitant flow through the commissure gap 114 lessens the opportunity for blood to stagnate behind the leaflet 106, which can lead to thrombus formation. The commissure gaps 114 are sized such that the resulting retrograde flow is minimal and does not otherwise increase strain on the patient's heart to pump blood through the conduit 102. The commissure gaps 114 may also increase the fluid flow at the commissure when the leaflets 106 are in the open position, such as, but not limited, to ensure that there is no entrapment of the blood between two closely opposed leaflet surfaces that may occur if no commissure gap 114 is provided.

FIG. 4B is an illustration of a cutting pattern 436 for a conduit 102 as used in a valved conduit prosthesis 100, in accordance with an embodiment. The cutting pattern 436 includes multiple separate slits 434 with the number of slits 434 corresponding to a number of leaflets (not shown) that will be coupled to the conduit 102. The cutting pattern 436 also includes a lateral cut 438 in the conduit 102 that allows for the conduit 102 to be split into two conduit components for assembly, a first conduit 102a and a second conduit 102b as shown in FIG. 4B. The lateral cut 438 is arranged near, adjacent to, or at a midpoint of a longitudinal portion of the slits 434. The lateral cut 438 and the slits 434 may be formed by laser cutting, hand cutting, or other similar methods. Although two slits 434 are shown in FIG. 4B, the cutting pattern 436 may have additional slits (e.g., three slits for a three leaflet 106 valve as in a tricuspid valve, four slits, etc.).

The slits 434 and the lateral cut 438 correspond to the number of leaflets that are coupled to the conduit 102. Similar to FIG. 4A, the slits 434 are separated by a land 112. The lands 112 correspond to separation between the leaflets 106 within the conduit 102 {e.g., at a commissure). When the leaflets 106 are closed {e.g., the valve is closed), there is a space, the commissure gap 114, between the leaflets as shown in FIG. 1. The commissure gaps 114 allow blood to wash-out regions behind the leaflets when the leaflets are in the closed position as noted previously. The retrograde flow lessens the opportunity for blood to stagnate behind the leaflet 106, which can lead to thrombus formation. The commissure gaps 114 are sized such that the resulting retrograde flow is minimal and does not otherwise increase strain on the patient's heart to pump blood through the conduit 102. The commissure gaps 114 may also increase the fluid flow at the commissure when the leaflets 106 are in the open position, such as, but not limited, to ensure that there is no entrapment of the blood between two closely opposed leaflet surfaces that may occur if no commissure gap 114 is provided.

FIG. 4C is an illustration of another example cutting pattern 440 for a conduit 102 as used in a valved conduit prosthesis, in accordance with an embodiment. The cutting pattern 440 includes multiple separate slits 434 with the number of slits corresponding to a number of leaflets (not shown) that will be coupled to the conduit 102. The cutting pattern 440 also includes a lateral cut 438 that is also a cut in the conduit 102 that allows for the conduit 102 to be cut into two conduit components for assembly as shown in FIG. 4C, which defines a first conduit iota and a second conduit 102b. The lateral cut 438 and the slits 434 may be formed by laser cutting, hand cutting, or other similar methods. Although two slits 434 are shown in FIG. 4B, the cutting pattern 440 may have additional slits (e.g., three slits for a tricuspid valve, four slits, etc.).

As noted above, the slits 434 and the lateral cut 438 correspond to the number of leaflets that will be coupled to the conduit 102. Similar to FIG. 4A, the slits 434 are separated by a land 112. The lands 112 correspond to the commissure gaps 114, that is the separation between the leaflets within the conduit 102 when the valve is closed (e.g., maintaining the commissure gap 114 when the leaflets are in the closed position).

FIG. 5 is a leaflet 106 that may be used in embodiments of a valved conduit prosthesis 100, in accordance with an embodiment. As shown in FIG. 5, the leaflet 106 may include multiple tabs 542 that extend from and define the leaflet belly 125. The tabs 542 may be formed by cutting slits in an edge of the leaflet 106. The leaflet 106 is formed from a thin sheet-like material as discussed below. As shown in further detail with reference to FIGS. 6A-C, the tabs 542 may be used to couple the leaflet 106 to the exterior surface 320 of the conduit 102, also as shown in FIG. 3.

In certain embodiments, the leaflet 106 includes alignment tabs 544, which are tabs 544 that are adjacent to the leaflet free edge 107. The alignment tabs 544 are formed in the same manner as the tabs 542. The alignment tabs 544, when present in the leaflet 106, are used to interface with the conduit 102 to assist in aligning the leaflet 106 at the commissure 116 for attachment to the conduit 102.

FIG. 6A is an illustration of an example step in attachment of a leaflet 106 to a conduit 102, in accordance with an embodiment. For ease of illustration, an outflow portion 214 of the conduit 102 is shown in FIG. 6A, however, as noted with reference to FIG. 4A, the conduit 102 and the leaflet 106 may be coupled together without separating the conduit 102 into multiple sections.

The leaflet 106 is aligned with a slit 434 in the conduit 102. As noted above, multiple leaflets 106 may be coupled to the conduit 102. For ease of illustration, FIGS. 6A-C illustrate attachment of a single leaflet 106 to the conduit 102. In certain embodiments, the conduit 102 includes an alignment line 646 that extends longitudinally along the conduit 102. The alignment line 646 facilitates aligning the leaflet 106 in the conduit 102. The leaflet 106, in certain embodiments, includes alignment tabs 544 that are arranged with respect to the alignment line 646 to ensure that the geometry and alignment of the leaflet 106 within the conduit 102 is proper. In other embodiments, the slit 434 at the commissure 116 is used to assist in alignment of the leaflet 106 to the conduit 102.

Once the leaflet 106 is aligned with the slit 434, the tabs 542 may be folded onto the exterior surface 306 of the conduit 102. As shown in FIG. 6B, the alignment tabs 544 may be folded so as to align with the alignment line 646.

As shown in FIG. 6C, the tabs 542 may be folded in different directions. Alternating tabs 542, for example, may be folded against the exterior surface 320 of the proximal (or inflow) portion 212 and the distal (or outflow) portion 214 of the conduit 102. The tabs 542 (and alignment tabs 544) also referred to as the conduit attachment section of the leaflet 106, which may be coupled to the exterior surface 320 of the conduit 102 by, for example, but not limited thereto, an adhesive, thermal bonding, or chemical bonding (e.g., as detailed above with reference to FIGS. 6A-6C). Tabs 542 of the leaflet 106 extend through the slit 434 {e.g., as shown in FIG. 3) in the conduit 102 defined between the proximal (or inflow) portion 212 and the distal (or outflow) portion 214. The remaining portions of the leaflet 106, the leaflet belly 125, extends into the conduit lumen 122 of the conduit 102 operable to function as a valve in the valved conduit prosthesis 100.

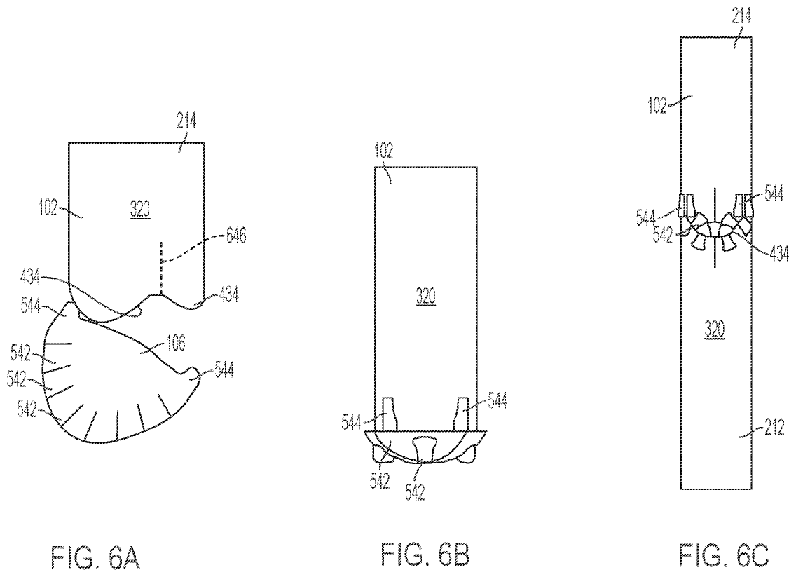

FIG. 7 is a cross-sectional illustration of another example valved conduit prosthesis 100, in accordance with an embodiment, wherein the inflow end 213 defines an outward taper 128 or is operable to be outwardly tapered. This outward taper 128 may facilitate the surgical attachment of the inflow end 213 to anatomy.

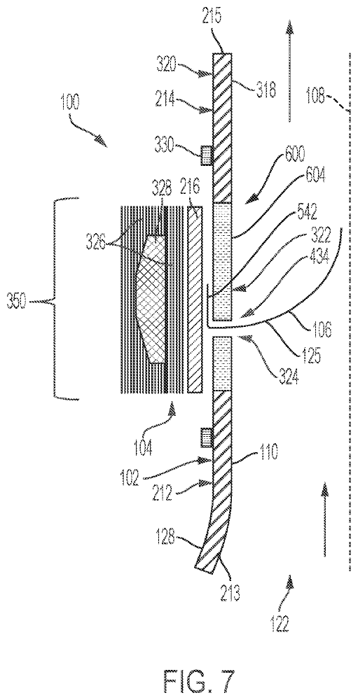

FIG. 8 is a cross-sectional view of another example valved conduit prosthesis 100, in accordance with an embodiment, wherein the inflow portion 212 is operable to be outwardly everted and rolled toward the valve structure 104 so as to define a sewing cuff 13o. A sewing cuff 130 may facilitate the surgical attachment of the inflow portion 212 to anatomy.

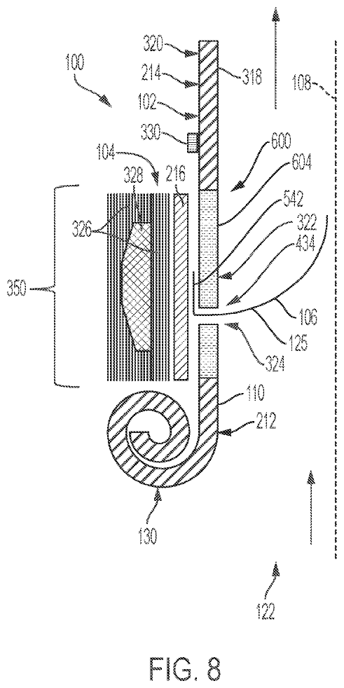

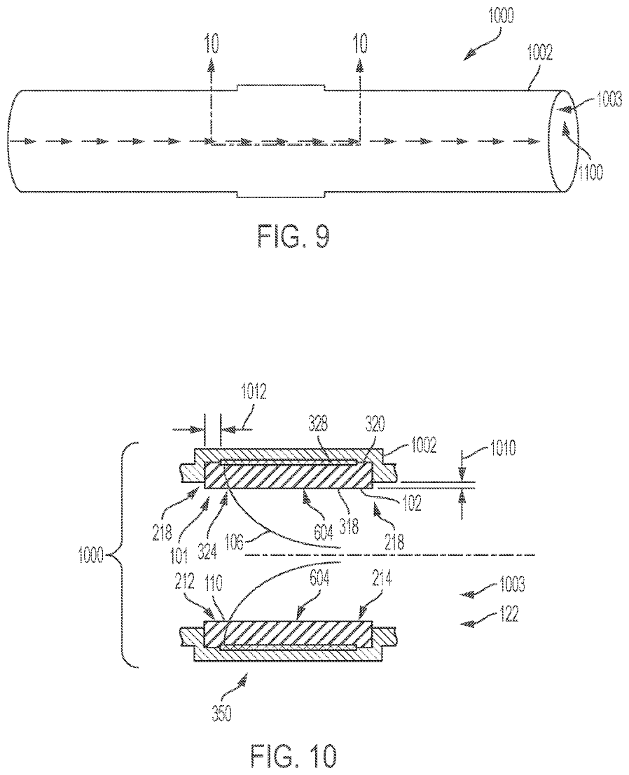

FIG. 9 is a side view and FIG. 10 is a longitudinal cross-sectional view of a valved conduit assembly moo, in accordance with an embodiment. This embodiment may be referred to as a valved conduit within a secondary conduit, or otherwise referred to as a valved conduit assembly woo. Essentially, these embodiments include any of the previous embodiments of the valved conduit prosthesis 100, or a portion of the valved conduit prosthesis 100 containing the valve region 350, which is hereinafter referred to as the valved conduit subassembly 101 as shown in FIGS. 10 and 11, that is disposed within a secondary conduit 1002. In accordance with an embodiment, and referring to FIG. 3, the valved conduit assembly moo/valved conduit subassembly 101 includes the valve region 350, which includes the densified portion 604 of the conduit 102, the leaflets 106, and the support frame 328. The conduit 102 is hereinafter referred to as the primary conduit 102 so as to differentiate from the secondary conduit 1002 into which the valved conduit subassembly 101 is disposed. The primary conduit 102 includes a primary conduit exterior surface 320 and a primary conduit interior surface 318 defining a primary conduit lumen 122. The primary conduit 102 is shorter in length than the secondary conduit 1002. The valved conduit subassembly 101/valve region 350 is disposed within a secondary conduit lumen 1003 of the secondary conduit 1002 and coupled to the secondary conduit inner surface 1100 of the secondary conduit 1002. In accordance with an embodiment, the secondary conduit 1002 is a tubular member having elastic properties such that the secondary conduit 1002 conforms to the outside profile of the valved conduit subassembly 101/valve region 350. The valved conduit subassembly 101/valve region 350 may be fixed within the secondary conduit 1002 by any of, for example, friction between the secondary conduit 1002 and the valve region 350, by a thermal bonding process, mechanical means, and adhesive. In other embodiments, the secondary conduit 1002 comprises a tape wrap construct that is wrapped into a tubular shape with the valved conduit subassembly 101/valve region 350 contained therein.

In the embodiment shown in FIG. 10, the support frame 328 is disposed between the primary conduit 102 and the secondary conduit 1002. It is understood that in other embodiments there may be no support frame 328 or the support frame may be disposed around the outer surface of the secondary conduit 1002 and positioned about a longitudinal axis coincident with the valve region 350 operable to provide radial support to the valve region 350.

FIG. 10 shows a step 218 or discontinuity of the secondary conduit inner surface 2013 defined by the inflow portion 212 of the valved conduit subassembly 101/valve region 350 and the outflow portion 214 of the valved conduit subassembly 101/valve region 350 due to the secondary conduit 1002 having a larger inner diameter compared with the inner diameter of the valved conduit subassembly 101/valve region 350. The step height low, and the distance between the step 218 and the leaflet 106, referred to as the upstream length 1012, where the leaflet 106 intersects the densified portion 604, are predetermined for a particular purpose. By way of example, the step 218 may have a step height low operable as a barrier to prevent pannus or other biological material from advancing into an upstream or downstream portion of the valved conduit subassembly 101/valve region 35o and advancing to the leaflet 106. In combination with the step height low, the upstream length 1012 has a dimension such that any pannus that is at the step 218 that may produce a flap beyond the step 218, it will not interfere with the leaflet 106. By way of example, the upstream length 1012 may have a dimension of 0.1 mm to 3 mm, and the step height low may have a dimension of 0 mm to 3 mm. By way of example, the valved conduit subassembly 101/valve region 350 may have an inner diameter of 16.5 mm and the secondary conduit may have an inner diameter of 18 mm, wherein the step height 1010 may have a nominal dimension of 0.8 mm. It is understood that the step height 1010 will present a trade-off as the step 218 becomes larger of the benefit of being a barrier to pannus overgrowth and a cause of flow disturbance.

It is understood that the step 218 may define a number of geometries. By way of example, as shown in FIG. 10, the step 218 is defined by 90-degree angles of the inflow portion 212 and the outflow portion 214 and with the secondary conduit inner surface 2012. It is understood that the step 218 may alternatively define a different geometry, such as, but not limited to, a fillet, a taper, and other curved or straight forms. By way of example, a relatively elastic secondary conduit 1002 may present a curved or rounded geometry as it is stretched and conforms to the shape of the valved conduit subassembly 101/valve region 350.

In embodiments of the valved conduit assembly moo, it is appreciated that if there is a leak at the opening 324, as best shown in FIG. 3, the fluid path remains within the lumen of the secondary conduit 1002. This is in contrast to the path formed in the valved conduit prosthesis 100 as presented in FIG. 3.

FIG. 11 shows a step 218 or discontinuity of the inner surface defined by the inflow portion 212 of the valve region 350 and the outflow portion 214 of the valve region 350 due to the secondary conduit 1002 having an elasticity that allows for conforming to the exterior surface 320 of the valved conduit subassembly 101/valve region 350 but leaves a step 218 at the leading and trailing edge of the inflow portion 212 and outflow portion 214. The inner diameter of the valve region 350 is the same as the inner diameter of the secondary conduit 1002 away from the leading and trailing edge of the inflow portion 212 and outflow portion 214, respectively. The size, depth and/or length of the step 218 or gap is predetermined for a particular purpose as provided above regarding, such as, but not limited to, pannus overgrowth prevention.

The valved conduit prostheses discussed herein are used to replace diseased anatomy in a surgical operation. Prior to implantation, the valved conduit prosthesis may be rinsed in saline and does not require pre-clotting. In accordance with one method of treatment, the valved conduit prosthesis is used as a replacement for an aortic valve and a portion of the ascending aorta, such as in an aortic root replacement. Implanting the valved conduit prosthesis includes identifying the inflow and outflow portions of the conduit, accessing the intended position with respect to the anatomy, and optionally trimming the inflow and or outflow conduit, while under moderate tension, to the appropriate length for implantation. The ascending aorta is sectioned and the inflow portion and/or inflow end of the valved conduit prosthesis is sutured or otherwise coupled to the left ventricle adjacent to or in the place of an excised aortic valve. The outflow portion and/or the outflow end of the valved conduit prosthesis is sutured to the sectioned ascending aorta. Coronary arteries are allowed to remain on the ascending aorta or they may be sutured to the outflow portion of the conduit and a flow path is provided from the conduit lumen to the coronary arteries.

In accordance with another method of treatment, the valved conduit prosthesis 100 may be a replacement of the native pulmonary valve or of a previously implanted pulmonary valved conduit prosthesis where partial or complete reconstruction of the right ventricular outflow tract and/or main pulmonary artery is desired. In certain instances, implantation of the valved conduit prosthesis includes identifying the inflow and outflow portions of the conduit, accessing the intended position with respect to the coronary arteries to assure there is no risk of coronary compression when implanted, and optionally trimming the inflow and or outflow conduit, while under moderate tension, to the appropriate length for implantation.

In accordance with another method of treating aortic valve disease by replacing the aortic root of a patient, the method comprises the steps of providing a valved conduit prosthesis 100 in accordance with embodiments herein and surgically implanting the valved conduit prosthesis 100. The method may further comprise identifying an inflow portion 212 and outflow portion 214 of the conduit 102; accessing the intended position with respect to anatomy; optionally trimming the inflow portion 212 and outflow portion 214 of the conduit to the appropriate length for implantation; optionally outwardly tapering the inflow end 213 or optionally everting and rolling the inflow portion 212 toward the valve structure 104 defining a sewing cuff 130; sectioning the ascending aorta; coupling the inflow portion 212 of the valved conduit prosthesis 100 to the left ventricle adjacent to or in the place of an excised aortic valve; and coupling the outflow portion 214 of the valved conduit prosthesis 100 to the sectioned ascending aorta. The method may further comprise coupling coronary arteries to the outflow portion 214 of the conduit 102; and establishing a flow path from the conduit lumen 122 to the coronary arteries. The method may further comprise coupling the coronary arteries 12 to a sinus defined by the outflow portion.

### Leaflet Material

The expanded fluoropolymer membrane can comprise any suitable microstructure, such as pores, for achieving the desired leaflet performance. Other biocompatible polymers which can be suitable for use in leaflet include but are not limited to the groups of urethanes, silicones (organopolysiloxanes), copolymers of silicon-urethane, styrene/isobutylene copolymers, polyisobutylene, polyethylene-co-poly(vinyl acetate), polyester copolymers, nylon copolymers, fluorinated hydrocarbon polymers and copolymers or mixtures of each of the foregoing.

In various examples, any of the leaflet 106 described herein (e.g., leaflet construct) may be formed of a biocompatible, synthetic material (e.g., including ePTFE and ePTFE composites, or other materials as desired). Other biocompatible polymers which can be suitable for use in synthetic leaflets include but are not limited to the groups of urethanes, silicones (organopolysiloxanes), copolymers of silicon-urethane, styrene/isobutylene copolymers, polyisobutylene, polyethylene-co-poly(vinyl acetate), polyester copolymers, nylon copolymers, fluorinated hydrocarbon polymers and copolymers or mixtures of each of the foregoing.

In other examples, such leaflet construct is formed of a natural material, such as repurposed tissue, including bovine tissue, porcine tissue, or the like.

As The term “elastomer” refers to a polymer or a mixture of polymers that has the ability to be stretched to at least 1.3 times its original length and to retract rapidly to approximately its original length when released.

The term “elastomeric material” refers to a polymer or a mixture of polymers that displays stretch and recovery properties similar to an elastomer, although not necessarily to the same degree of stretch and/or recovery.

The term “non-elastomeric material” refers to a polymer or a mixture of polymers that displays stretch and recovery properties not similar to either an elastomer or elastomeric material, that is, considered not an elastomer or elastomeric material as is generally known.

In accordance with embodiments herein, the leaflet 106 comprises a composite material having at least one porous synthetic polymer membrane layer having a plurality of pores and/or spaces and an elastomer and/or an elastomeric material and/or a non-elastomeric material filling the pores and/or spaces of the at least one synthetic polymer membrane layer. In accordance with other examples, the leaflet 106 further comprises a layer of an elastomer and/or an elastomeric material and/or a non-elastomeric material on the composite material. In accordance with examples, the composite material comprises porous synthetic polymer membrane by weight in a range of 10% to 90%.

An example of a porous synthetic polymer membrane includes expanded fluoropolymer membrane having a node and fibril structure defining the pores and/or spaces. In some examples, the expanded fluoropolymer membrane is expanded polytetrafluoroethylene (ePTFE) membrane. Another example of porous synthetic polymer membrane includes microporous polyethylene membrane and expanded polyethylene.

Examples of an elastomer and/or an elastomeric material and/or a non-elastomeric material include, but are not limited to, copolymers of tetrafluoroethylene and perfluoromethyl vinyl ether (TFE/PMVE copolymer), (per)fluoroalkylvinylethers (PAVE), urethanes, silicones (organopolysiloxanes), copolymers of silicon-urethane, styrene/isobutylene copolymers, polyisobutylene, polyethylene-co-poly(vinyl acetate), polyester copolymers, nylon copolymers, fluorinated hydrocarbon polymers and copolymers or mixtures of each of the foregoing. In some examples, the TFE/PMVE copolymer is an elastomer comprising between 60 and 20 weight percent tetrafluoroethylene and respectively between 40 and 80 weight percent perfluoromethyl vinyl ether. In some examples, the TFE/PMVE copolymer is an elastomeric material comprising between 67 and 61 weight percent tetrafluoroethylene and respectively between 33 and 39 weight percent perfluoromethyl vinyl ether. In some examples, the TFE/PMVE copolymer is a non-elastomeric material comprising between 73 and 68 weight percent tetrafluoroethylene and respectively between 27 and 32 weight percent perfluoromethyl vinyl ether. The TFE and PMVE components of the TFE-PMVE copolymer are presented in wt %. For reference, the wt % of PMVE of 40, 33-39, and 27-32 corresponds to a mol % of 29, 23-28, and 18-22, respectively.

In some examples, the TFE-PMVE copolymer exhibits elastomer, elastomeric, and/or non-elastomeric properties.

In some examples, the composite material further comprises a layer or coating of TFE-PMVE copolymer comprising from 73 to 68 weight percent tetrafluoroethylene and respectively from 27 to 32 weight percent perfluoromethyl vinyl ether.

In some examples, the leaflet the leaflet 106 is an expanded polytetrafluoroethylene (ePTFE) membrane having been imbibed with TFE-PMVE copolymer comprising from 60 to 20 weight percent tetrafluoroethylene and respectively from 40 to 80 weight percent perfluoromethyl vinyl ether, the leaflet 106 further including a coating of TFE-PMVE copolymer comprising from 73 to 68 weight percent tetrafluoroethylene and respectively 27 to 32 weight percent perfluoromethyl vinyl ether on the blood-contacting surfaces.

As discussed above, the elastomer and/or an elastomeric material and/or a non-elastomeric material may be combined with the expanded fluoropolymer membrane such that the elastomer and/or the elastomeric material and/or the non-elastomeric material occupies substantially all of the void space or pores within the expanded fluoropolymer membrane.

Although some examples of suitable leaflet materials have been provided, the foregoing examples are not meant to be read in a limiting sense, and additional or alternative materials are contemplated.

Inventive features of this disclosure have been described above both generically and with regard to specific embodiments. It will be apparent to those skilled in the art that various modifications and variations can be made in the embodiments without departing from the scope of the disclosure. Thus, it is intended that the embodiments cover the modifications and variations of this disclosure provided they come within the scope of the appended claims and their equivalents.

What is claimed is:

1. A valved conduit prosthesis comprising:
a secondary conduit having a secondary conduit inner surface defining a secondary conduit lumen therethrough; and
a valved conduit subassembly including:
a primary conduit having a primary conduit exterior surface and a primary conduit interior surface defining a primary conduit lumen and a slot therethrough; and
at least one leaflet having an external portion coupled to the primary conduit exterior surface and an internal portion arranged within the primary conduit lumen so as to be operable as a one-way valve, the leaflet defining a valve structure,
wherein the valved conduit subassembly is located within the secondary conduit lumen and coupled to the secondary conduit inner surface.

2. The valved conduit prosthesis of claim 1, wherein the external portion of the at least one leaflet is adhered to the primary conduit exterior surface by adhesive, thermal bonding, or chemical bonding.

3. The valved conduit prosthesis of claim 1, wherein the external portion of the at least one leaflet is coupled to the primary conduit exterior surface by a layer of adhesive film.

4. The valved conduit prosthesis of claim 1, wherein the valved conduit subassembly defines at least one step with the secondary conduit inner surface.

5. The valved conduit prosthesis of claim 4, wherein the at least one step is defined by the secondary conduit having a larger inner diameter compared with the inner diameter of the valved conduit subassembly.

6. The valved conduit prosthesis of claim 4, wherein the step may have a step height defined by the primary conduit inner surface and the secondary conduit inner surface at the step, wherein the step height is operable as a barrier to prevent pannus or other biological material from advancing into an upstream or downstream portion of the valved conduit subassembly.

7. The valved conduit prosthesis of claim 4, wherein the valved conduit subassembly defines an upstream length between the leaflet and the step, wherein the upstream length has a dimension of 0.1 mm to 3 mm, and a step height has a dimension of 0 mm to 3 mm.

8. The valved conduit prosthesis of claim 1, wherein the valved conduit has an inflow portion defining an inflow end and an outflow portion defining an outflow end, wherein the at least one leaflet is coupled to the primary conduit is operable to open to allow flow from the inflow end to pass through the outflow end in antegrade flow conditions, and are operable to close to restrict flow from flowing from the outflow end through the inflow end in retrograde flow conditions.

9. The valved conduit prosthesis of claim 1, wherein the at least one leaflet comprises a composite material including a porous synthetic fluoropolymer membrane defining pores and an elastomer or elastomeric material filling the pores, and optionally TFE-PMVE copolymer comprising from 27 to 32 weight percent perfluoromethyl vinyl ether and respectively from 73 to 68 weight percent tetrafluoroethylene on at least a portion of the composite material, and optionally, the elastomer or elastomeric material comprises a TFE-PMVE copolymer, and optionally, the porous synthetic fluoropolymer membrane is ePTFE.

10. The valved conduit prosthesis of claim 1, wherein the at least one leaflet includes more than one leaflets, and the more than one leaflets are separated from one another within the primary conduit inner surface of the primary conduit by commissure gaps.

11. The valved conduit prosthesis of claim 10, wherein the at least one leaflet is more than one leaflet and the primary conduit includes lands separating each leaflet at a conduit attachment section of each of the leaflets to form the commissure gaps between the leaflets within the primary conduit inner surface of the primary conduit.

12. The valved conduit prosthesis of claim 1, wherein the valved conduit subassembly is attached to the secondary conduit inner surface by an adhesive, thermal bonding, or chemical bonding.

13. A method of treating aortic valve disease by replacing an aortic root, comprising the steps of:
providing a valved conduit prosthesis of claim 1; and
surgically implanting the valved conduit prosthesis.
